(19) United States
(12) Patent Application Publication
(10) Pub. No.: US 2011/0195117 A1
(43) Pub. Date: Aug. 11, 2011

(54) CONTROLLED RELEASE COMPOSITIONS OF ROPINIROLE

(75) Inventors: Arvind Mohan Upadhyay, Maharashtra (IN); Girish Bang, Maharashtra (IN); Nikhil Malewar, Maharashtra (IN); Makrand Avachat, Maharashtra (IN)

(73) Assignee: Lupin Limited, Mumbai (IN)

(21) Appl. No.: 13/061,410

(22) PCT Filed: Sep. 1, 2009
(86) PCT No.: PCT/IN09/00479

§ 371 Date: Feb. 28, 2011

(30) Foreign Application Priority Data
Sep. 1, 2008 (IN) 1510/KOL/2008

(51) Int. Cl. A61K 9/48; A61K 9/00; A61K 31/4045; A61P 25/16

(52) U.S. Cl. 424/452; 424/400; 514/418

(57) ABSTRACT

A novel oral controlled release pharmaceutical composition comprising a therapeutically effective amount of active substance, ropinirole or a pharmaceutically acceptable salt(s) or enantiomer(s) or polymorph(s) or hydrate(s) thereof, one or more controlled release agent(s), optionally one or more pharmaceutically acceptable excipient(s) and an extended release coating, wherein the said composition provides controlled release of the active agent with reduced initial burst release.

### FIELD OF INVENTION

This invention relates to novel controlled release pharmaceutical compositions of ropinirole and the process of preparing them.

### BACKGROUND OF THE INVENTION

Ropinirole was first disclosed in U.S. Pat. No. 4,452,808. The US Patent '808 also discloses the pharmaceutical compositions of ropinirole. The U.S. Pat. No. 4,824,860 discloses method of treating Parkinson's using ropinirole. The U.S. Pat. No. 4,997,954 discloses process for the preparation of ropinirole.

Ropinirole (marketed under the brand names Requip and Ropark in extended release form as Requip XL) is an non-ergoline dopamine agonist. Ropinirole hydrochloride (4-(2-di-n-propylaminoethyl)-2(3H)-indolone hydrochloride) is approved in most territories for the treatment of Parkinson's disease and has also been disclosed as being of potential use in the treatment of a variety of other conditions, such as Restless Legs Syndrome (RLS; Ekbom Newsletter, July 1997), fibromyalgia (U.S. Pat. No. 6,277,875), acute CNS injury (Medico, M. et al., (2002), European Neuropsychopharmacology 12, 187-194), various sleep related disorders such as apneas, hypopneas and snoring events (Saletu, M. et al., (2000), Neuropsychobiology 41, 190-199) and chronic fatigue syndrome (U.S. Pat. No. 6,300,365).

Ropinirole has a high water solubility (133 mg/ml at 25° C.). This can lead to difficulty in providing a slow release rate from a formulation and problems in controlling the initial burst of drug from such a formulation. Drugs with high solubility in water (for example, greater than 100 mg/ml) can be difficult to formulate into a controlled release oral dosage form. Solubility is a driving force for a drug substance to dissolve in water; the greater the solubility the greater the rate of dissolution, when all other factors are maintained constant. A further problem with highly water soluble drugs formulated into a controlled release dosage form is that a significant and variable “burst” of drug can occur from these systems.

In the prior art, many techniques have been used to provide controlled and extended-release pharmaceutical compositions of ropinirole in order to maintain therapeutic levels of medicaments and to minimize the effects of missed doses of drugs caused by a lack of patient compliance. The commercially available extended release composition of ropinirole is REQUIP XL.

Some of controlled release compositions for ropinirole described in prior art are as follows:

US20070264336 (Jagotech) describes a multilayer controlled release tablet compositions, which consists of 0.05-20% ropinirole, hydrophilic polymer, which swell and/or gel, lipophilic substances and 5-50% adjuvant substances.
US20030180359 (Jagotech) describes a multilayer controlled release tablet compositions, which consists of active, hydrophilic polymeric substance, which swell and/or gel upon contact with aqueous liquids, lipophilic substances and adjuvant substances with a hydrophilic and lipophilic substance ratio is in the range from 10:1 to 0.5:1.
US20070059365 (Smithkline) describes a novel controlled release dosage form of ropinirole with specific pharmacokinetics which is used in the treatment of diseases, which can prevent or disturb sleep, particularly Restless Legs Syndrome (RLS).

The prior art patent teaches multilayer composition with a barrier layer, which uses complicated technology with low yield, however there still is a need of better ropinirole controlled release compositions.

Thus the present invention provides a novel controlled release composition of ropinirole or its pharmaceutically acceptable salts thereof, which provide freedom from burst effect, associated with formulation of controlled release of highly water-soluble drug which are easy to manufacture, comparatively cheaper, bioequivalent and having higher manufacturing yield compared to marketed formulation.

### OBJECTS OF THE INVENTION

The object of the present invention is a novel oral controlled release pharmaceutical composition comprising a therapeutically effective amount of active substance, ropinirole or a pharmaceutically acceptable salt(s) or enantiomer(s) or polymorph(s) or hydrate(s) thereof, one or more controlled release agent(s), optionally one or more pharmaceutically acceptable excipient(s) and an extended release coating, wherein the said composition provides controlled release of the active agent with reduced initial burst release.

Another object of the present invention is a novel oral controlled release pharmaceutical composition comprising a therapeutically effective amount of ropinirole or a pharmaceutically acceptable salt(s) or enantiomer(s) or polymorph(s) or hydrate(s) thereof, one or more controlled release agent(s), optionally one or pharmaceutically acceptable excipient(s), and an extended release coating, wherein the said composition provides controlled release of the active agent with reduced initial burst release, characterized in that the controlled release pharmaceutical composition is bioequivalent to marketed controlled release formulation.

Another object of the present invention is a novel oral controlled release pharmaceutical composition comprising a therapeutically effective amount of ropinirole or a pharmaceutically acceptable salt(s) or enantiomer(s) or polymorph(s) or hydrate(s) thereof, optionally one or pharmaceutically acceptable excipient(s) and an extended release coating, wherein the said dosage form provides controlled release of the active agent with reduced initial burst release, wherein about 0-15% of drug is released in about 1 hour, about 25-50% of drug released in about 4 hours, about 50-75% of drug released in about 9 hours, and greater than about 85% of drug released in about 24 hours.

### DETAILED DESCRIPTION OF THE INVENTION

The present invention is directed towards a novel controlled release oral pharmaceutical composition comprising (1) a core comprising ropinirole or its pharmaceutically acceptable salts, one or more release controlling polymer(s) (2) coating with an extended release polymer(s) wherein the said composition provides controlled release of the active agent with reduced initial burst release. The formulation of the present invention provides controlled release of ropinirole with no burst release which is commonly associated with highly water soluble drug along with controlled release action for extended period which is further easy to manufacture, simple and gives a higher yield with a dissolution pattern similar to the marketed formulation.

“Controlled release,” means drug delivery system releasing the drug at a predetermined rate, locally or systemically, for a specified period of time. Controlled release can be used interchangeably with prolonged release, programmed release, timed release, extended release, sustained release and other such dosage forms.

“Therapeutically effective amount” means that the amount of active agent, which halts or reduces the progress of the condition being treated or which otherwise completely or partly cures or acts palliatively on the condition. A person skilled in the art can easily determine such an amount by routine experimentation and with an undue burden.

The term “Ropinirole” as used is the invention is meant to cover Ropinirole in the form of freebase or its pharmaceutically acceptable salt(s), hydrate(s), solvate(s) and physiologically functional derivative(s) and precursors thereof. The term also includes all polymorphic forms, whether crystalline or amorphous.

As used herein, “pharmaceutically acceptable salts” refer to derivatives of the Ropinirole wherein Ropinirole is modified by reacting it with an acid or base as needed to form an ionically bound pair. Examples of pharmaceutically acceptable salts include conventional non-toxic salts or the quaternary ammonium salt of the parent compound formed, for example, from non-toxic inorganic or organic acids. Suitable non-toxic salts include those derived from inorganic acids such as hydrochloric, hydrobromic, sulfuric, sulfamic, phosphoric, nitric, and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, benzoic, salicylic, sulfanilic, fumaric, oxalic, isethionic, and others known to those of ordinarily skilled in the art. The most preferred salt of Ropinirole for the present invention is hydrochloride salt

By “pharmaceutically acceptable” is meant a carrier comprised of a material that is not biologically or otherwise undesirable.

The “inert core” according to the present invention is a pharmaceutically substance which is inactive in relation to the active ingredient, that is to say, it does not react with the active ingredient in the conditions used in such a way that there is decomposition thereof, and it may be composed of for example sugar, saccharose, starch, cellulose and mixtures thereof.

The release controlling polymers can be hydrophilic, lipophilic or combination thereof.

The hydrophilic rate-controlling polymer includes but are not limited to celluloses or their salts or derivatives thereof, hydroxyethylcellulose, hydroxypropyl cellulose, hydroxypropyl methylcellulose, sodium carboxymethyl cellulose, alginic acid or their salts and derivatives thereof, carbomer (Carbopol™), polyethyleneoxide, xanthan gum, guar gum, locust bean gum, poly vinyl acetate, polyvinyl alcohol. Preferably the rate-controlling polymer is hydroxypropylmethylcellulose.

The hydrophilic polymer is present in percentage between 1% to 95% w/w of the total formulation. Preferably the hydrophilic polymer is present between about 25% to about 95% w/w of the total formulation.

The lipophilic rate controlling agent in matrix includes but are not limited to hydrogenated vegetable oil, but other suitable agents include purified grades of beeswax; fatty acids; long chain fatty alcohols, such as cetyl alcohol, myristyl alcohol, and stearyl alcohol; glycerides such as glyceryl esters of fatty acids like glyceryl monostearate, glyceryl distearate, glyceryl behenate, glyceryl esters of hydrogenated castor oil and the like; oils such as mineral oil and the like, or acetylated glycerides; ethyl cellulose, stearic acid, paraffin, carnauba wax, talc; and the stearate salts such as calcium, magnesium, zinc and other materials known to one of ordinary skill in the art.

The lipoophilic polymer is present in percentage between 1% to 95% w/w of the total formulation. Preferably the lipoophilic polymer is present between about 15% to about 55% w/w of the total formulation.

Pharmaceutically acceptable excipients include but are not limited to binders, diluents, lubricants, glidants and surface-active agents. One excipient can perform more than one function.

Binders include, but are not limited to, starches such as potato starch, wheat starch, corn starch; microcrystalline cellulose such as products known under the registered trade marks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations there of and other materials known to one of ordinary skill in the art and mixtures thereof.

Fillers or diluents, which include, but are not limited to carbohydrates, confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like and other materials known to one of ordinary skill in the art and combinations thereof.

Lubricants may be selected from, but are not limited to, those conventionally known in the art such as Mg, Al or Ca or Zn stearate, polyethylene glycol, glyceryl behenate, mineral oil, sodium stearyl fumarate, stearic acid, hydrogenated vegetable oil and talc, and the like and other materials known to one of ordinary skill in the art and combinations thereof.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art and combinations thereof.

The pharmaceutical composition according to present invention may also comprise a disintegrant which may be included in all or part of the oral dosage form to ensure rapid disintegration of the dosage form or part of the dosage from (for example, one of the layers in a bilayer tablet) after administration. Suitable disintegrants include, but are not limited to: alginic acid, carboxymethylcellulose calcium, carboxymethylcellulose sodium, croscarmellose sodium, crospovidone, guar gum, magnesium aluminum silicate, sodium alginate, sodium starch glycolate and starches and other materials known to one of ordinary skill in the art and combinations thereof.

The pharmaceutical dosage form of the invention can optionally have one or more coatings such as film coating, sugar coating, extended release coating, enteric coating, bioadhesive coating and other coatings known in the art. These coatings help pharmaceutical formulations to release the drug at and for the required time. The most preferable coating is extended release coating. The amount of coating is usually within about 1% w/w to about 25% w/w of the total composition weight.

These coating comprises one or more excipients selected from the group comprising coating agents, opacifiers, fillers, polishing agents, colouring agents, antitacking agents and the like.

Coating agents which are useful in the coating process, include, but are not limited to, water insoluble waxes and polymers such as polymethylacrylates (for example Eudragit polymers, Trade Mark) or water insoluble celluloses, particularly ethylcellulose. Optionally, water-soluble polymers such as polyvinylpyrrolidone or water-soluble celluloses such as hydroxypropylmethylcellulose or hydroxypropylcellulose may be included. Optionally other water-soluble agents such as polysorbate 80 may be added. polysaccharides such as maltodextrin, alkyl celluloses such as methyl or ethyl cellulose, hydroxyalkylcelluloses (e.g. hydroxypropylcellulose or hydroxypropylmethylcelluloses); polyvinylpyrrolidone, acacia, corn, sucrose, gelatin, shellac, cellulose acetate phthalate, lipids, synthetic resins, acrylic polymers, opadry, polyvinyl alcohol (PVA), copolymers of vinylpyrrolidone and vinyl acetate (e.g. marketed under the brand name of Plasdone) and polymers based on methacrylic acid such as those marketed under the brand name of Eudragit. These may be applied from aqueous or non-aqueous systems or combinations of aqueous and non-aqueous systems as appropriate. Additives can be included along with the film formers to obtain satisfactory films. These additives can include plasticizers such as dibutyl phthalate, triethyl citrate, polyethylene glycol (PEG) and the like, antitacking agents such as talc, stearic acid, magnesium stearate and colloidal silicon dioxide and the like, surfactants such as polysorbates and sodium lauryl sulphate, fillers such as talc, precipitated calcium carbonate, Polishing agents such as Beeswax, carnauba wax, synthetic chlorinated wax and opacifying agents such as titanium dioxide and the like. All these excipients can be used at levels well known to the persons skilled in the art.

Non-permeable coatings of insoluble polymers, e.g., cellulose acetate, ethylcellulose, can be used as enteric coatings for delayed/modified release (DR/MR) by inclusion of soluble pore formers in the coating, e.g., HPMC, PEG, PVA, sugars, salts, detergents, triethyl citrate, triacetin, etc.

Pharmaceutical dosage forms of the invention can be coated by a wide variety of methods. Suitable methods include compression coating, coating in a fluidized bed or a pan and hot melt (extrusion) coating. Such methods are well known to those skilled in the art.

The most preferable extended release coating according to the present invention is a coating using a combination of ethylcellulose and HPMC. Thus the extended release coating according to the present invention helps in controlling the initial burst of the drug or it can also be used to provide the controlled release of ropinirole throughout the release period.

The burst of highly water-soluble drug is the initial rapid release of drug that occurs from oral controlled release dosage forms when first contacting fluid, such as gastric fluids, prior to release controlling mechanisms of the dosage form.

The basic mechanism by which the drug is released is that initially water penetrates through the tablet surfaces and swells. At this stage, drug is not released from the pharmaceutical composition thereby reducing the initial burst release of drug. Later, the coating at the edges of the tablet weakens upon swelling of the tablet, and slowly the drug starts releasing from the edges thereby controlling the release of the drug from the composition.

The amount of ropinirole present, inclusive of pharmaceutically acceptable salts thereof, may be up to about 24.0 mg, preferably from about 0.75 mg to about 12.0 mg, measured as the amount of ropinirole base present, that is excluding any amount of acid (for example, hydrochloric acid, HCl) added to form any ropinirole salts.

The pharmaceutical composition according to present invention may be tablet, capsules, pellet, bead, spheroids, microcapsules, minitablet, tablet in tablet, powder, granules, pellets/granules/beads in capsules and others.

The various embodiments of the present invention can be assembled in several different ways.

In one embodiment, the tablet comprises a solid core comprising ropinirole or a pharmaceutically acceptable salt(s) or enantiomer(s) or polymorph(s) thereof, one or more hydrophilic rate controlling polymer(s) and at least one pharmaceutically acceptable excipient(s). This hydrophilic matrix tablet is then coated with an extended release coating.

In one embodiment, the tablet comprises a solid core comprising ropinirole or a pharmaceutically acceptable salt(s) or enantiomer(s) or polymorph(s) thereof, one or more lipophilic rate controlling polymer(s) and at least one pharmaceutically acceptable excipient(s). This lipophilic matrix tablet can optionally be coated with an extended release coating.

In one embodiment, the tablet comprises a solid core comprising ropinirole or a pharmaceutically acceptable salt(s) or enantiomer(s) or polymorph(s) thereof and at least one pharmaceutically acceptable excipient(s) with an extended release coating.

In one embodiment, the tablet comprises a solid core comprising the active ingredient ropinirole or a pharmaceutically acceptable salt(s) or enantiomer(s) or polymorph(s) thereof, one or more hydrophilic and lipophilic polymer(s), and at least one pharmaceutically acceptable excipient(s), with or without an extended release coating.

In one embodiment, the bilayer pharmaceutical composition comprising, a) first layer which comprises, a therapeutically effective amount of ropinirole or a pharmaceutically acceptable salt(s) or enantiomer(s) or polymorph(s) thereof, one or more release controlling agent(s), one or more pharmaceutically acceptable excipient(s); and b) another layer which comprises one or more release controlling agent(s) and one or more pharmaceutically acceptable excipient(s) wherein the bilayer composition is optionally coated with an extended release coating.

The pharmaceutical composition of the invention can be formed by various methods known in the art such as by dry granulation, wet granulation (aqueous, non-aqueous, hydroalcoholic), melt granulation, direct compression, dry granulation, double compression, extrusion spheronization, layering and the like.

The solvent(s) used in wet granulation in the present invention include all the solvents well known in the art or their mixtures thereof.

The pharmaceutical composition according to present invention can be made by several different methods.

In one embodiment the invention can be prepared by a method which comprises preparing controlled release oral pharmaceutical composition comprising (1) preparing granules comprising Ropinirole, or a pharmaceutically acceptable salt(s) or enantiomer(s) or polymorph(s) thereof, one or more pharmaceutically acceptable excipient(s) and one or more release controlling agent(s) using aqueous, non-aqueous or hydroalcoholic technique (2) further processing, lubricating and then compressing the granules to prepare the controlled release solid dosage form.

In one embodiment the invention can be prepared by a method which comprises preparing controlled release oral pharmaceutical composition comprising (1) preparing granules comprising Ropinirole, or a pharmaceutically acceptable salt(s) or enantiomer(s) or polymorph(s) thereof, one or more pharmaceutically acceptable excipient(s) (2) further processing, lubricating and then compressing the granules to prepare the controlled release tablet (3) Further the tablet is coated with an extended release coating.

In one embodiment the invention can be prepared by a method which comprises preparing controlled release oral pharmaceutical composition comprising (1) preparing granules comprising Ropinirole, or a pharmaceutically acceptable salt(s) or enantiomer(s) or polymorph(s) thereof, one or more pharmaceutically acceptable excipient(s) and one or more control releasing agent(s) by melt granulation (2) further processing, lubricating and then compressing the granules to prepare the controlled release tablet.

In one embodiment the invention can be prepared by a method which comprises preparing controlled release oral pharmaceutical composition comprising (1) preparing pellets comprising Ropinirole, or a pharmaceutically acceptable salt(s) or enantiomer(s) or polymorph(s) thereof, one or more pharmaceutically acceptable excipient(s) and one or more control releasing agent(s) by melt extrusion (2) further processing, lubricating and then compressing the granules to prepare the controlled release tablet which can optionally be given an extended release coating. (3) The coated pellets can further be compressed into tablet or filled into capsules.

In another embodiment the invention can be prepared by a method comprising: a first granulation comprising Ropinirole or its pharmaceutically acceptable salt(s), polymorph(s), solvate(s), hydrate(s) thereof and one or more release controlling material; and a second granulation comprising one or more release controlling material which is the same or different than the one or more release controlling material of the first granulation and optionally Ropinirole or its pharmaceutically acceptable salts, polymorphs, solvates, hydrates thereof, compressing both the granules to form a tablet which can optionally be further coated with an extended release coating.

In another embodiment the invention can be prepared by a method comprising an inert core (a) applying a first layer comprising ropinirole or its pharmaceutically acceptable salt(s), polymorph(s), solvate(s), hydrate(s) thereof and optionally one or more release controlling material with one or more pharmaceutically acceptable excipient(s) and optionally (b) one or more separating layer(s) and optionally (c) extended releasing coating. The pellets obtained can optionally be given an overcoating layer. The pellets can be filled in capsules or compressed into tablets.

In the most preferred embodiment, the process of making the pharmaceutical formulation of the invention comprises as described below:
i) Blending the active agent, releasing controlling agent(s), pharmaceutically acceptable excipient(s),
ii) subjecting the blend to granulation.
iii) drying and seiving the granulated blend to obtain granules
iv) Blending the granules obtained in the above step with extragranular excipient(s)
v) Compressing the blend of step (iv) to form the solid oral dosage form.
vi) the dosage form coated with an extended release coating.

The pharmaceutical composition according to the present invention is easy to prepare, gives a higher yield and bioequivalent compared to the marketed formulation.

The following examples are illustrative of the present invention, and the examples should not be considered as limiting the scope of this invention in any way, as these examples and other equivalents thereof will become apparent to those versed in the art, in the light of the present disclosure, and the accompanying claims.

### EXAMPLES

### Example 1

| | | (mg/unit |
|---|---|---|
| S. No | Name of the Ingredients | dose) |
| | CORE | |
| 1 | Ropinirole hydrochloride | 2.00 |
| | equivalent to Ropinirole | |
| 2 | Lactose monohydrate/ | 92.25 |
| | microcrystalline cellulose | |
| 3 | Colloidal silicon dioxide | 2.00 |
| 4 | Povidone | 3.00 |
| 5 | Purified water | QS |
| 6 | Magnesium stearate | 0.75 |
| | EXTENDED RELEASE COATING | |
| 1 | Ethyl cellulose | 2.25 |
| 2 | Hypromellose | 0.75 |
| 3 | Isopropyl alcohol | QS |
| 4 | Dichloromethane | QS |

Brief Manufacturing Procedure:
1. Mix Ropinirole and geometrically and sift through a suitable sieve.
2. Sift Colloidal silicon dioxide and povidone through a suitable sieve and mix with the powder mix of step 1.
3. Granulate the powder mix of step 2 with purified water and dry the granules at suitable temperature and pass them to get suitable size granules. Lubricate the blend with Magnesium stearate and compress into tablets.
4. Dissolve Ethyl cellulose and Hypromellose in the Isopropyl alcohol and Dichloromethane solution.
5. Coat the tablets of step 4 with the above coating composition to get the desired profile

### Example 2

| | | (mg/unit |
|---|---|---|
| S. No | Name of the Ingredients | dose) |
| 1 | Ropinirole hydrochloride | 3.00 |
| | equivalent to Ropinirole | |
| 2 | Hypromellose | 361 |
| 3 | Lactose monohydrate/ | 14 |
| | microcrystalline cellulose | |
| 4 | Colloidal silicon dioxide | 4 |
| 5 | Povidone | 12 |
| 6 | Aqueous/Non-aqueous/ | QS |
| | hydroalcohilic solvent | |
| 7 | Colloidal silicon dioxide | 4 |
| 8 | Magnesium stearate | 2 |
| | EXTENDED RELEASE COATING | |
| 1 | Ethyl cellulose | 8.4 |
| 2 | Hypromellose | 3.6 |
| 3 | Isopropyl alcohol | QS |
| 4 | Dichloromethane | QS |

Brief Manufacturing Procedure:
1. Mix Ropinirole and Lactose monohydrate geometrically and sift through a suitable sieve and mix with hypromellose.
2. Sift Colloidal silicon dioxide and povidone through a suitable sieve and mix with the powder mix of step 1.
3. Granulate the powder mix of step 2 with suitable binder solution and dry the granules at suitable temperature and pass them to get suitable size granules.
4. Lubricate the blend with colloidal silicon dioxide and magnesium stearate and compress into tablets.
5. Dissolve Ethyl cellulose and Hypromellose in the Isopropyl alcohol and Dichloromethane solution.
6. Coat the tablets of step 4 with the above coating composition to get the desired profile.

### Example 3

| | | (mg/unit |
|---|---|---|
| S. No | Name of the Ingredients | dose) |
| 1 | Ropinirole hydrochloride | 4.00 |
| | equivalent to Ropinirole | |
| 2 | Hypromellose | 120 |
| 3 | Hydrogenated castor oil | 120 |
| 4 | Lactose monohydrate | 152 |
| 5 | Colloidal silicon dioxide | 4 |
| 6 | Magnesium stearate | 2 |

Brief Manufacturing Procedure:
1. Mix Ropinirole, Lactose monohydrate, hypromellose and hydrogenated castor oil at suitable temperature with continuous stirring.
2. Maintain the temperature of the melt mass for a sufficient period of time.
3. Cool the melted mass at room temperature until a hard mass is obtained.
4. Sift the mass through a suitable sieve.
5. Lubricate the blend with colloidal silicon dioxide and magnesium stearate and compress into tablets.

### Example 4

| | | (mg/unit |
|---|---|---|
| S. No | Name of the Ingredients | dose) |
| 1 | Ropinirole hydrochloride | 8.00 |
| | equivalent to Ropinirole | |
| 2 | Hydrogenated castor oil | 25.00 |
| 3 | Lactose monohydrate | 65.25 |
| 4 | Colloidal silicon dioxide | 1.00 |
| 5 | Magnesium stearate | 0.75 |

Brief Manufacturing Procedure:
1. Mix Ropinirole, Lactose monohydrate and hydrogenated castor oil at suitable temperature with continuous stirring.
2. Maintain the temperature of the melt mass for a sufficient period of time.
3. Cool the melted mass at room temperature until a hard mass is obtained.
4. Sift the mass through a suitable sieve.
5. Lubricate the blend with colloidal silicon dioxide and magnesium stearate and compress into tablets.

### Example 5

| | | (mg/unit |
|---|---|---|
| S. No | Name of the Ingredients | dose) |
| 1 | Ropinirole hydrochloride | 2.00 |
| | equivalent to Ropinirole | |
| 2 | Microcrystalline cellulose | 135.20 |
| 3 | Hypromellose | 2.8 |
| 4 | Purified water | QS |
| | Total: | |
| | EXTENDED RELEASE COATING | |
| 1 | Ethyl cellulose | 9.94 |
| 2 | Hypromellose | 4.06 |
| 3 | Triethyl citrate | 1.4 |
| 4 | Talc | 4.6 |
| 5 | Isopropyl alcohol | QS |
| 6 | Dichloromethane | QS |

Brief Manufacturing Procedure:
1. Mix Ropinirole with microcrystalline cellulose and sift them through a suitable sieve.
2. Prepare hypromellose solution in suitable quantity of water
3. Granulate the blend of step 1 with binder solution of step 2.
4. Extrudate the wet mass through 1 mm sieve using extruder and spheronize using spheronizer
5. Dry the pellets at a suitable temperature and size them to suitable fractions.
6. Disperse ethyl cellulose, hypromellose, Triethyl cellulose and talc in isopropyl alcohol and dichloromethane solution.
7. Coat pellets with the above coating composition.
8. The coated pellets can either be filled in the capsules or compressed as tablets.

### Example 6

| | | (mg/unit |
|---|---|---|
| S. No | Name of the Ingredients | dose) |
| | Layer I | |
| 1 | Ropinirole hydrochloride | 2.00 |
| | equivalent to Ropinirole | |
| 2 | Hypromellose | 20.00 |
| 3 | Hydrogenated castor oil | 20.00 |
| 4 | Lactose monohydrate | 16.80 |
| 5 | Colloidal silicon dioxide | 0.60 |
| 6 | Magnesium stearate | 0.60 |
| | Layer II | |
| 1 | Hypromellose | 200.00 |
| 2 | Lactose monohydrate | 133.20 |
| 3 | Colloidal silicon dioxide | 3.40 |
| 4 | Magnesium stearate | 3.40 |
| | EXTENDED RELEASE COATING | |
| 1 | Ethyl cellulose | 8.40 |
| 2 | Hypromellose | 3.60 |
| 3 | Isopropyl alcohol | 68.40 |
| 4 | Dichloromethane | 159.60 |

Brief Manufacturing Procedure:
1. Mix Ropinirole, Lactose monohydrate, hypromellose and hydrogenated castor oil of layer I at suitable temperature with continuous stirring.
2. Maintain the temperature of the melt mass for a sufficient period of time.
3. Cool the melted mass at room temperature until a hard mass is obtained.
4. Sift the mass through a suitable sieve.
5. Lubricate the blend with colloidal silicon dioxide and magnesium stearate.
6. Mix hypromellose, lactose monohydrate, colloidal silicon dioxide and magnesium stearate of layer II and sift through a suitable sieve.
7. Compress the blend of step 5 and step 6 into a tablet in tablet.
8. Dissolve Ethyl cellulose and Hypromellose in the Isopropyl alcohol and Dichloromethane solution.
9. Coat the tablets of step 7 with the above coating composition to get the desired profile.

### Example 7

| | | (mg/unit |
|---|---|---|
| S. No | Name of the Ingredients | dose) |
| 1 | Ropinirole hydrochloride | 3.00 |
| | equivalent to Ropinirole | |
| 2 | Hypromellose | 120.00 |
| 3 | Hydrogenated castor oil | 120.00 |
| 4 | Lactose monohydrate | 151.00 |
| 5 | Colloidal silicon dioxide | 4.00 |
| 6 | Magnesium stearate | 2.00 |
| | Layer II | |
| 1 | Hypromellose | 60.00 |
| 2 | Lactose monohydrate | 136.00 |
| 3 | Colloidal silicon dioxide | 2.00 |
| 4 | Magnesium stearate | 2.00 |
| | EXTENDED RELEASE COATING | |
| 1 | Ethyl cellulose | 8.40 |
| 2 | Hypromellose | 3.60 |
| 3 | Isopropyl alcohol | QS |
| 4 | Dichloromethane | QS |

Brief Manufacturing Procedure:
1. Mix Ropinirole, Lactose monohydrate, hypromellose and hydrogenated castor oil at suitable temperature with continuous stirring.
2. Maintain the temperature of the melt mass for a sufficient period of time.
3. Cool the melted mass at room temperature until a hard mass is obtained.
4. Sift the mass through a suitable sieve.
5. Lubricate the blend with colloidal silicon dioxide and magnesium stearate.
6. Mix hypromellose, lactose monohydrate, colloidal silicon dioxide and magnesium stearate of layer II and sift through a suitable sieve.
7. Compress the blend of step 5 and step 6 into a bilayered tablet.
8. Dissolve Ethyl cellulose and Hypromellose in the Isopropyl alcohol and Dichloromethane solution.
9. Coat the tablets of step 7 with the above coating composition to get the desired profile.

### Example 8

| | | (mg/unit |
|---|---|---|
| S. No. | Name of the Ingredients | dose) |
| 1. | Ropinirole hydrochloride | 2.00 |
| | equivalent to Ropinirole | |
| 2. | Hypromellose | 300.00 |
| 3. | Lactose monohydrate | 100.50 |
| 4. | Microcrystalline cellulose | 60.00 |
| 5. | Colloidal silicon dioxide | 5.00 |
| 6. | Povidone | 20.00 |
| 7. | Methanol | QS |
| 8. | Colloidal silicon dioxide | 10.00 |
| 9. | Magnesium stearate | 2.50 |

Brief Manufacturing Procedure:
1. Mix Ropinirole, Hypromellose, Lactose Monohydrate, Microcrystalline Cellulose, Colloidal Silicon Dioxide, Povidone together in low/high shear mixer and granulate with methanol.
2. Dry and size the granules
3. Mix sized granules with Colloidal Silicon Dioxide
4. Lubricate the granules with Magnesium Stearate.
5. Compress the tablets
6. Coat the tablets with EC: Hypromellose using organic solvent to give the coat build up of 2.5% w/w of the tablet weight.

### Example 9

| | | (mg/unit |
|---|---|---|
| S. No. | Name of the Ingredients | dose) |
| 1. | Ropinirole hydrochloride | 2.00 |
| | equivalent to Ropinirole | |
| 2. | Hypromellose | 200.00 |
| 3. | Microcrystalline cellulose | 160.50 |
| 4. | Colloidal silicon dioxide | 5.00 |
| 5. | Povidone | 20.00 |
| 6. | Methanol | QS |
| 7. | Hypromellose | 100.00 |
| 8. | Colloidal silicon dioxide | 10.00 |
| 9. | Magnesium stearate | 2.50 |

Brief Manufacturing Procedure:

1. Mix intragranular Hypromellose, Lactose Monohydrate, Microcrystalline Cellulose, Colloidal Silicon Dioxide, and Povidone together in low/high shear mixer and granulate with methanol.
2. Dry and size the granules.
3. Mix sized granules with Colloidal Silicon Dioxide and extragranular Hypromellose
4. Lubricate the granules with Magnesium Stearate.
5. Compress the tablets.
6. Coat the tablets with EC: Hypromellose using organic solvent to give the coat build up of the tablet weight.

### Example 10

| | | (mg/unit |
|---|---|---|
| S. No | Name of the Ingredients | dose) |
| 1 | Ropinirole hydrochloride | 2.00 |
| | equivalent to Ropinirole | |
| 2 | Hypromellose | 300 |
| 3 | Microcrystalline cellulose | 160.22 |
| 4 | Colloidal silicon dioxide | 15 |
| 5 | Povidone | 22.5 |
| 6 | Methyl alcohol | QS |
| 7 | Colloidal silicon dioxide | 4 |
| 8 | Magnesium stearate | 2 |
| 9 | Hypermellose | 150 |
| | Coating | |
| 10 | Ethyl cellulose | 7.5 |
| 11 | Hypermellose | 7.5 |
| 12 | IPA | QS |
| 13 | Methylene chloride | QS |

1. Sift Hypromellose, Microcrystalline Cellulose Colloidal Silicon Dioxide and Povidone through specific s.s.sieve.
2. Load approximately half quantity of excipients of step 2 into rapid mixer granulator (RMG) followed by excipients of step 1. Mix for 15 minutes on slow speed agitator and chopper off.
3. Dissolve Ropinorole Hydrochloride in Methyl Alcohol to prepare the solution in suitable container.
4. Add the Ropinorole Hydrochloride solution into the powder mass of step 2 by gradual addition in RMG. Rinse the container with Methyl Alcohol and add the rinsing to the powder mass in RMG. Add extra Methyl Alcohol to achieve the granules of suitable consistency if required.
5. Load the wet mass in fluid bed dryer & air dry.
6. Sift the dried granules through specific sieve and mill the retained dried granules though 1.00 mm screen.
7. Load the approximately half quantity of dried granules of step 6 in conta blender followed by Mixing for 20 minutes.
8. Sift Magnesium Stearate through specific ss sieve and add to the conta blender. Mix for three minutes.
9. Compress the lubricated blend of step Busing suitable punch.
10. Dissolve ethylcellulose and hypermellose in Isopropyl alcohol and dichloromethane solution.
11. Coat the tablets of step 91th step 10.

In-Vitro Dissolution Study

In-vitro test is used to measure release rate of the active agent from a composition. Any suitable dissolution medium can be used for the in-vitro dissolution of the present invention. The tablet composition of example 10 was placed in the USP dissolution apparatus I using 0.01 N HCl for first three hours followed by 6.8 phosphate buffer as dissolution media and dissolution was periodically measured.

TABLE 1
InVitro dissolution of example 10

| Time (hr) | Cumulative % Drug Release |
|---|---|
| 1 | About 0-15 |
| 4 | About 25-50 |
| 9 | About 50-75 |
| 24 | More than about 85 |

TABLE 2
InVitro dissolution of example 10 without coating

| Time (hr) | Cumulative % Drug Release |
|---|---|
| 1 | About 15-25 |
| 4 | About 35-60 |
| 9 | About 60-80 |
| 24 | More than about 85 |

In-Vivo Bioequivalence Study

Open Label, balanced, randomized, single-dose, two-treatment, two-sequence, two-period crossover relative bioequivalence study of Ropinirole hydrochloride Extended Release tablets 2 mg of Lupin Limited, India Comparing with that of Requip—XL Tablets 2 mg of GlaxoSmithkline in 42 healthy adult human male subjects under fed and fasting conditions. The study was designed to demonstrate the similar clinical efficacy compared to Requip-XL® (2 MG).

The in-vivo bioequivalence study shows the results as shown in the table below:

TABLE 3
Comparative pharmacokinetic parameters (Geometric
Least Squares Mean) of present invention vs
RequipXL ® (2 mg) (Fed study)

| | AUC (0-t) | AUC (0-∞) | Cₘₐₓ |
|---|---|---|---|
| | pg · hr/ml | pg · hr/ml | pg/ml |
| Example 10(T) | 31115.488 | 31666.596 | 1665.089 |
| Requip- XL 2 mg(R) | 31768.535 | 32328.077 | 1645.874 |
| T/R | 97.94 | 97.95 | 101.17 |

TABLE 4
Comparative pharmacokinetic parameters (Geometric
Least Squares Mean) of present invention vs RequipXL ® (2
mg) (Fasting study)

| | AUC (0-t) | AUC (0-∞) | Cₘₐₓ |
|---|---|---|---|
| | pg · hr/ml | pg · hr/ml | pg/ml |
| Example 10(T) | 23089.339 | 23668.500 | 1323.202 |
| Requip- XL 2 mg(R) | 24337.254 | 25686.343 | 1335.803 |
| T/R | 94.87 | 92.14 | 99.06 |

1. A novel oral controlled release pharmaceutical composition comprising a therapeutically effective amount of active substance, ropinirole or a pharmaceutically acceptable salt(s) or enantiomer(s) or polymorph(s) or hydrate(s) thereof, one or more controlled release agent(s), optionally one or more pharmaceutically acceptable excipient(s) and an extended release coating, wherein the said composition provides controlled release of the active agent with reduced initial burst release.

2. The novel oral controlled release pharmaceutical composition as in claim 1 is wherein the pharmaceutical composition is granules, capsules, tablet, pellets, minitablets, microcapsules, tablet in capsules, granules in capsules, pellets in capsules.

3. The novel oral controlled release pharmaceutical composition as in claim 1, wherein the control releasing agent(s) is hydrophilic, lipophilic or combinations thereof.

4. The novel oral controlled release pharmaceutical composition as in claim 1, wherein pharmaceutically acceptable excipients are selected from the group comprising binders, diluents, lubricants, surfactants and glidants.

5. The novel oral controlled release pharmaceutical composition as in claim 1 wherein the extended release coating comprises one or more water-soluble or water-insoluble polymers or combinations thereof.

6. A novel oral controlled release pharmaceutical composition comprising a therapeutically effective amount of ropinirole or a pharmaceutically acceptable salt(s) or enantiomer(s) or polymorph(s) or hydrate(s) thereof, one or more controlled release agent(s), optionally one or pharmaceutically acceptable excipient(s), and an extended release coating, wherein the said composition provides controlled release of the active agent with reduced initial burst release, characterized in that the controlled release pharmaceutical composition is bioequivalent to marketed controlled release formulation.

7. A novel oral controlled release pharmaceutical composition comprising a therapeutically effective amount of ropinirole or a pharmaceutically acceptable salt(s) or enantiomer(s) or polymorph(s) or hydrate(s) thereof, optionally one or pharmaceutically acceptable excipient(s) and an extended release coating, wherein the said dosage form provides controlled release of the active agent with reduced initial burst release, wherein about 0-15% of drug is released in about 1 hour, about 25-50% of drug released in about 4 hours, about 50-75% of drug released in about 9 hours, and greater than about 85% of drug released in about 24 hours.
